(19) United States
(12) Patent Application Publication
(10) Pub. No.: US 2010/0036249 A1
(43) Pub. Date: Feb. 11, 2010

(54) METHOD AND APPARATUS FOR MULTILINE COLOR FLOW AND ANGIO ULTRASOUND IMAGING

(75) Inventor: David W. Clark, Windham, NH (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/516,957

(22) PCT Filed: Dec. 3, 2007
(86) PCT No.: PCT/IB2007/054904

§ 371 Date: May 29, 2009

Related U.S. Application Data
(60) Provisional application No. 60/868,370, filed on Dec. 4, 2006.

(51) Int. Cl. A61B 8/14

(52) U.S. Cl. 600/443

(57) ABSTRACT

A method for multiline ultrasound imaging comprises implementing multiline beamforming with a number of ensembles (52,54,56,58). Each ensemble includes a sequence (64,66,68,70,72,74) of transmit beams (T) of a given transmit direction and a first multiple of receive beams (R) per transmit beam. The method further includes constructing an overlap multiline image (50) at a frame rate equivalent to a second multiple non-overlapping multiline. The second multiple is a multiple different from the first multiple.

The present embodiments relate generally to medical systems and more particularly, to a method and apparatus for multiline colorflow and angio ultrasound imaging.

Ultrasound imaging has been widely used to observe tissue structures within a human body, such as the heart structures, the abdominal organs, the fetus, and the vascular system. Ultrasound imaging systems include a transducer array connected to multiple channel transmit and receive beamformers applying electrical pulses to the individual transducers in a predetermined timing sequence to generate transmit beams that propagate in predetermined directions from the array.

As the transmit beams pass through the body, portions of the acoustic energy are scattered back to the transducer array from tissue structures having different acoustic characteristics. The receive transducers (which may be the transmit transducers operating in the receive mode) convert the scattered pressure pulses into corresponding RF signals that are provided to the receive beamformer. Due to different distances to the individual transducers, the scattered sound waves arrive at the individual transducers at different times, and thus the RF signals have different phases.

The receive beamformer has a plurality of processing channels with compensating delay elements connected to a summer. The receive beamformer uses a delay value for each channel and collect echoes scattered from a selected focal point. Consequently, when delayed signals are summed, a strong signal is produced from signals corresponding to this point, but signals arriving from different points, corresponding to different times, have random phase relationships and thus destructively interfere. Furthermore, the beamformer selects the relative delays that control the orientation of the receive beam with respect to the transducer array. Thus, the receive beamformer can dynamically steer the receive beams that have desired orientations and focus them at desired depths. In this way, the ultrasound system acquires echo data.

Ultrasound imaging can include different types of ultrasound, for example, color flow ultrasound and Color Power Angio (CPA) ultrasound. Color flow ultrasound detects blood velocity by estimating the average phase shift between echoes from successive transmits in an ensemble (packet) in the same direction, after strong, slow moving tissue echoes are attenuated with a high-pass clutter filter. Color Power Angio (CPA) ultrasound is similar, but displays the log power of the clutter filtered echoes.

Signal to noise ratio in color flow ultrasound and angio ultrasound can be improved by using a larger ensemble size. However, using a larger ensemble size undesirably reduces the ultrasound frame rate. Slower velocities can be imaged by interleaving different directions with the ensemble to reduce the pulse repetition frequency (PRF) in each direction without degrading the frame rate. Frame rate can be improved by beamforming multiple (typically 2 or 4) slightly different receive directions for each transmit beam. The receive beams are usually oversteered away from the transmit beam direction to make the round-trip beam position correct.

With 2× parallel ultrasound imaging, the receive beams of a scan have nominally identical signal to noise ratio, since the receive beams are equidistant from the transmit direction. However, with 4× multiline imaging (i.e., in a 4×1 planar scan), the outer receive beams have lower signal to noise ratio than the inner receive beams in view of the fact that the outer receive beams are at a different distance from the transmit direction than the inner receive beams. If the receive gain is the same for outer and inner beams, then the receive beam signal will be weaker in the outer beams, thereby causing a 4-line periodic pattern in the color signal. If the receive gain is increased in the outer beams to equalize the signal strength, then the noise will be stronger in the outer beams, causing a 4-line periodic pattern in the background noise.

Techniques have been proposed for grayscale imaging where RF signals from the same receive direction but different transmit directions are combined to reduce multiline artifacts. However, these techniques are not applicable to color flow and angio, because RF combining depends on having negligible motion between the two transmit times. Not only do color flow and angio inherently look at motion, but the ensemble size increases the time between geometrically adjacent transmits. In general, the decorrelation of the moving blood echo is shorter than the ensemble.

What is needed is a technique for using greater than 2× multiline in color flow and angio without the artifacts caused by varying signal to noise ratio. Accordingly, an improved method and system for overcoming the problems in the art is desired.

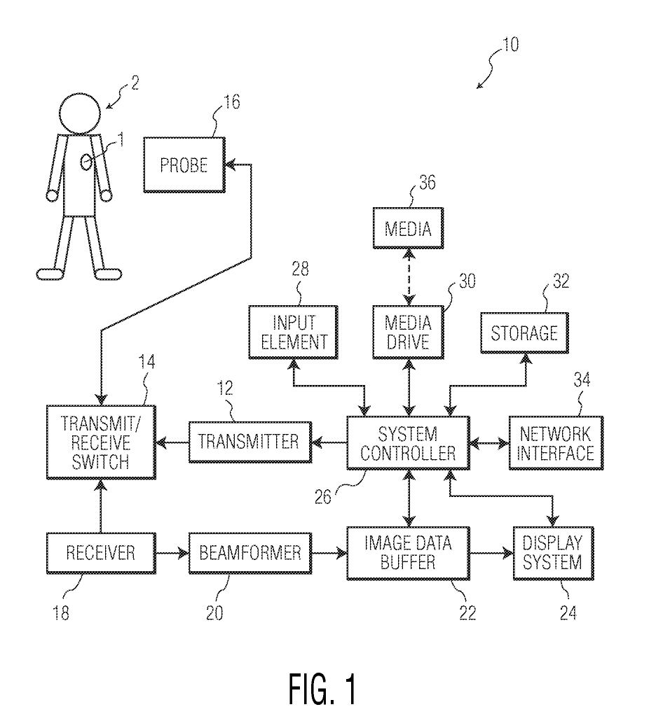

FIG. 1 is a block diagram view of a system for multiline ultrasound imaging according to one embodiment of the present disclosure;

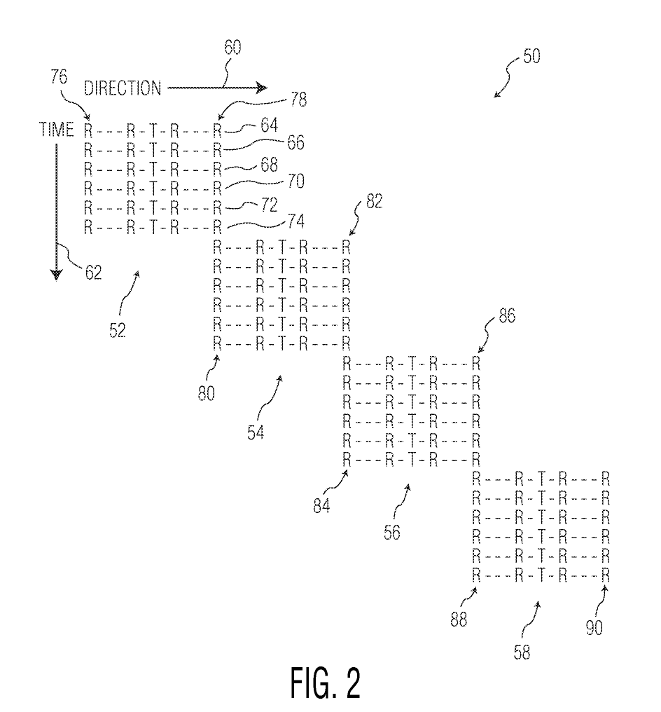

FIG. 2 is a diagram view illustrating transmit (T) and round-trip (R) directions for several ensembles, with no interleaving, of the multiline imaging method according to one embodiment of the present disclosure; and

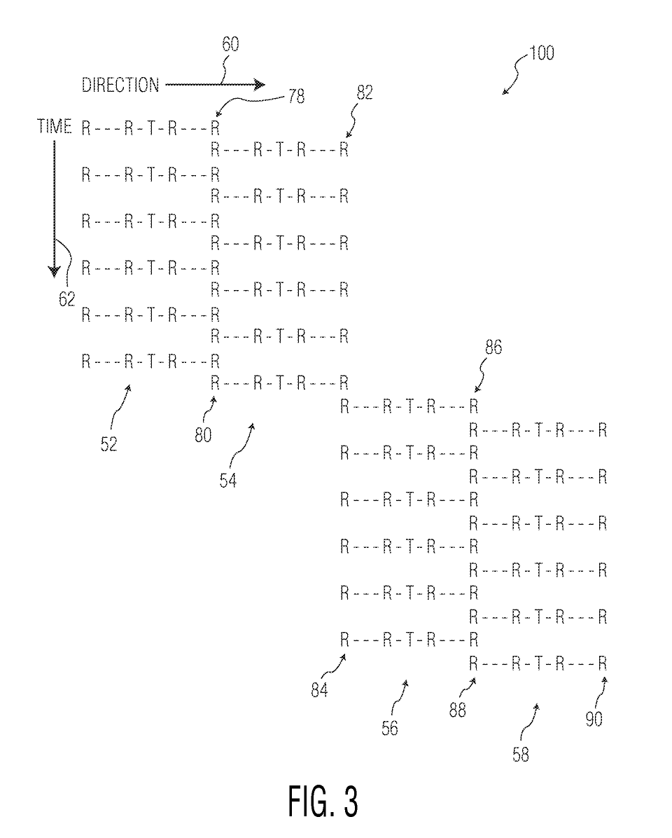

FIG. 3 is a diagram view illustrating transmit (T) and round-trip (R) directions for several ensembles, with interleaving by a factor of two (2), of the multiline imaging method according to another embodiment of the present disclosure.

In the figures, like reference numerals refer to like elements. In addition, it is to be noted that the figures may not be drawn to scale.

FIG. 1 is a block diagram view of a multiline ultrasound imaging system 10 suitable for implementing the various embodiments of the present disclosure. An ultrasound transmitter 12 is coupled through a transmit/receive (T/R) switch 14 to a transducer array or probe 16. Transducer array 16 comprises any suitable array of transducer elements for performing scanning in connection with the embodiments of the present disclosure. The transducer array 16 transmits ultrasound energy into a region being imaged and receives scattered ultrasound energy, or echos, from various structures and organs, for example the heart 1 within a patient's body 2. The transmitter 12 includes a transmit beamformer. By appropriately delaying the pulses applied to each transducer element by transmitter 12, the transmitter transmits a focused ultrasound beam along a desired transmit scan line.

The transducer array 16 couples to an ultrasound receiver 18 through T/R switch 14. Scattered ultrasound energy from a given point within the patient's body is received by the transducer elements at different times. The transducer elements convert the received ultrasound energy to received electrical signals which are amplified by receiver 18 and are supplied to a receive beamformer 20. The signals from each transducer element are individually delayed and then are summed by the beamformer 20 to provide a beamformer signal that is a representation of the scattered ultrasound energy level along a given receive scan line. The delays applied to the received signals may be varied in a suitable manner during reception of ultrasound energy to effect dynamic focusing. The process is repeated for multiple scan lines to provide signals for generating an image of a region of interest in the patient's body. In one embodiment, the transducer array can comprise a two-dimensional array, whereby the receive scan lines can be steered in azimuth and in elevation to form a three-dimensional scan pattern. The beamformer 20 may, for example, be a digital beamformer configured to perform various steps and/or functions in the multiline ultrasound imaging method according to the embodiments of the present disclosure.

Beamformer signals are stored in an image data buffer 22 which, as described below, stores image data for different segments of an image volume. The image data is output from image data buffer 22 to a display system 24 which generates an image of the region of interest from the image data. The display system 24 may include a scan converter which converts sector scan signals from beamformer 20 to conventional raster scan display signals.

A system controller 26 provides overall control of the multiline ultrasound imaging system. The system controller 26 performs timing and control functions and can include a microprocessor and associated memory. In one embodiment, system controller 26 comprises any suitable computer and/or control unit that can be configured for performing the various functionalities as discussed herein with respect to the method for multiline ultrasound imaging according to the various embodiments. Furthermore, programming of the system controller 26, for performing the methods according to the embodiments of the present disclosure as discussed herein, can be accomplished with use of suitable programming techniques. In addition, an electrocardiogram (ECG) device (not shown) can be used in connection with the multiline ultrasound imaging system of the present disclosure, wherein the ECG device includes use of ECG electrodes for being attached to a subject or patient. The ECG device supplies ECG waveforms to system controller 26 for synchronizing imaging to the patient's cardiac cycle, as necessary, for a given imaging procedure.

Multiline ultrasound imaging system 10 further includes input element 28, media drive 30, storage 32, and network interface 34, each coupled to system controller 26 for performing functions to be discussed further herein below. Input element 28 can include any suitable input device, for example, a keyboard, mouse, or other suitable input device or devices, for enabling user input to the multiline ultrasound imaging system. Media drive 30 includes any suitable media drive, for interfacing with one or more different types of media (36). For example, media drive 30 may include an optical read-write drive such as any one of a DVD-RAM, DVD+-RW or CD-RW drive. Media drive 30 may also include a read-write disc drive, such as a floppy drive. Still further, media drive 30 may include a drive suitable for reading and writing to a SmartMedia™, CompactFlash™, Memory Stick™, or other type of storage device presently known or developed in the future.

In addition, storage 32 comprises any suitable computer storage, such as a hard disk drive, for storing computer programs and data as discussed herein with respect to the embodiments of the present disclosure. Furthermore, network interface 34 is coupled to the system controller 26 for enabling system controller 26 to access a network, such as, an intranet, the Internet, an extranet, or other computer network.

In the embodiments of the present disclosure, the computer readable media preferably includes any computer readable media suitable for using in the method and apparatus of multiline ultrasound imaging according to the embodiments of the present disclosure. For example, media 36 can comprise a writable or re-writable CD, DVD, DVD-RAM, or other similar computer readable media. Media 36 may also comprise, for example, a SmartMedia™, CompactFlash™, Memory Stick™, or other type of storage device presently known or developed in the future. Still further, the computer readable media may include a network communication media. Examples of network communication media include, for example, an intranet, the Internet, or an extranet.

Refering now to FIG. 2, the figure illustrates a view 50 of transmit (T) and round-trip (R) directions for several ensembles, with no interleaving, of the multiline imaging method according to one embodiment of the present disclosure. In particular, four ensembles 52, 54, 56, and 58 are shown as a function of direction 60 horizontally and as a function of time 62 vertically. Each ensemble includes a sequence of transmit beams of a given transmit direction and a first multiple of receive beams per transmit beam. For example, for ensemble 52, the sequence of transmit beams of the given transmit direction and first multiple of received beams per transmit beam are indicated by reference numerals 64, 66, 68, 70, 72, and 74. Ensembles 54, 56, and 58 have similar sequences of transmit beams for respective directions. In addition, in FIG. 2, the first multiple is illustrated as 4×.

According to one embodiment of the present disclosure, a method for multiline ultrasound imaging comprises implementing multiline beamforming with a number of ensembles. Each ensemble includes a sequence of transmit beams of a given transmit direction and a first multiple of receive beams per transmit beam. The method further includes constructing an overlap multiline image at a frame rate equivalent to a second multiple non-overlapping multiline. The second multiple is a multiple different from the first multiple. In the overlap multiline image, the receive beams of a first ensemble overlap, in a predetermined manner, receive beams of an adjacent ensemble. In one embodiment, the second multiple is a multiple less than the first multiple. For example, the first multiple can comprise four times (4×) multiline and the second multiple can comprise three times (3×) multiline. In other words, in the embodiment where the receive beamforming is 4× multiline for all ensembles, the result of the overlap of outer beam directions of adjacent ones of the ensembles, in a predetermined manner, produces a frame rate equivalent to 3× non-overlapping multiline. That is, the beamforming is 4×, but the resulting image is 3×. Each sequence of an ensemble includes outer round-trip beams, for example, as indicated by reference numerals 76 and 78 for ensemble 52, 80 and 82 for ensemble 54, 84 and 86 for ensemble 56, and 88 and 90 for ensemble 58.

In one embodiment, implementing the multiline beamforming includes: configuring corresponding ones of the outer round-trip beams of adjacent ensembles to occur along a same direction (i.e., overlap); and combining sample correlations derived from the outer round-trip beams of the adjacent ensembles along the same direction. For example, as shown in FIG. 2, outer round-trip beams 80 are configured to occur along a same direction as outer round-trip beams 78 of ensemble 52. In a similar manner, outer round-trip beams 84 are configured to occur along a same direction as outer round-trip beams 82 of ensemble 54. Furthermore, outer round-trip beams 88 are configured to occur along a same direction as outer round-trip beams 86 of ensemble 56.

The multiline ultrasound imaging method further comprises processing ensemble data for outer round-trip beams separately for each ensemble. In such an instance, the ensemble data for outer round-trip beams of a first ensemble having a first transmit direction is processed separately from ensemble data for outer round-trip beams of a second ensemble having a second transmit direction. The method further comprises performing clutter filtering and sample correlation with no mixing of data from different transmit directions of respective ensembles. Performing clutter filtering and sample correlation with no mixing of data from different transmit directions includes processing outer beams of ensemble data for a first transmit direction separately from outer beams of ensemble data for a second transmit direction, the second transmit direction being different from the first transmit direction.

According to another embodiment, the multiline ultrasound imaging method further includes adjusting various beamforming parameters (e.g., ensemble size, beam spacing, region of interest, etc.) to ensure that the frame rate is adequate to observe the hemodynamics (i.e., average velocity, amplitude, turbulence, etc.) in the desired region of interest. Since there are multiple ensembles per frame, the hemodynamic changes between adjacent ensembles are even smaller than what the user has determined is acceptable between frames.

The method according to an embodiment of the present disclosure further comprises changing a (beamforming) transmit direction between successive frames of ensembles, wherein each frame includes of a predetermined number of ensembles. Transmit directions of a current frame are configured to occur where the outer round-trip beams of a previous frame occur.

According to one embodiment of the present disclosure, a multiline ultrasound imaging method utilizes 4× multiline beamforming to construct a 3× multiline color flow or angio image. The beamformer places outer round-trip beams from adjacent transmit directions in the same direction. Then the sample correlations from the same-direction outer beams are combined, producing a signal to noise improvement similar to a double-size ensemble, which compensates for the reduced signal to noise of outer beams. This works even though the RF blood signal decorrelates over the ensemble, and it works whether or not interleaving is used.

The ensemble data are processed in a suitable manner, including use of clutter filtering, and computing sample correlations as x(n)*conj(x(n−1) ). For outer round-trip beams, the ensemble data is processed separately for each transmit direction, wherein the clutter filtering and sample correlation advantageously does not mix data from different transmit directions.

Subsequent to the clutter filtering and the sample correlation of the outer round-trip beams, the sample correlations for the outer beams in the same direction are combined, that is, as if they came from a larger ensemble. The color flow or angio algorithms continue, using twice as many sample correlations for every third geometric line. The larger number of sample correlations compensates for the lower signal to noise ratio of the outer beams.

Because the sample correlations measure lag-1 phase changes rather than absolute phase, there is no requirement for coherence from one ensemble to the next (or even coherence through an ensemble). There is only the assumption that the hemodynamics does not change significantly from one ensemble to the next, but that is an assumption with any color flow imaging.

For further reduction of multiline artifacts, the beamforming directions can be changed on successive frames, so that (for example) the transmit directions are where the outer beams were on the previous frame. In such an instance, a slight amount of temporal filtering (persistence) will attenuate any residual multiline artifact.

Referring now to FIG. 3, the figure illustrates a view 100 of transmit (T) and round-trip (R) directions for several ensembles, with interleaving by a factor of two (2), of the multiline imaging method according to another embodiment of the present disclosure. Note that the technique according to the embodiments of the present disclosure is not limited to an interleave factor of two (2), since this is illustrative of only one example of how the overlapping of outer beams can be combined with interleaving. In particular, four ensembles 52, 54, 56, and 58 are shown as a function of direction 60 horizontally and as a function of time 62 vertically. Each ensemble includes a sequence of transmit beams of a given transmit direction and a first multiple of receive beams per transmit beam. In addition, in the embodiment of FIG. 3, while the receive beamforming is 4× multiline for all ensembles, the interleaved overlap of outer beam directions of adjacent ones of the ensembles, as shown, produces a frame rate equivalent to 3× non-overlapping multiline. In other words, the beamforming is 4×, but the resulting image is 3×.

In the embodiment of FIG. 3, configuring corresponding ones of the outer round-trip beams of adjacent ensembles to occur in the same direction includes interleaving corresponding ones of the outer round-trip beams 78 of a first ensemble 52 with corresponding ones of the outer round-trip beams 80 of a second ensemble 54 adjacent the first ensemble, by an interleaving factor of two (2). Configuring corresponding ones of the outer round-trip beams of adjacent ensembles to occur in the same direction still further includes not interleaving corresponding ones of the outer round-trip beams 82 of a second ensemble 54 with corresponding ones of the outer round-trip beams 84 of a third ensemble 56 adjacent the second ensemble. Configuring corresponding ones of the outer round-trip beams of adjacent ensembles to occur in the same direction still further includes interleaving corresponding ones of the outer round-trip beams 86 of a third ensemble 56 with corresponding ones of the outer round-trip beams 88 of a fourth ensemble 58 adjacent the second ensemble, by an interleaving factor of two (2).

According to another embodiment, a system for multiline ultrasound imaging, comprises means for implementing multiline beamforming with a number of ensembles, each ensemble including a sequence of transmit beams of a given transmit direction and a first multiple of receive beams per transmit beam; and means for constructing an overlap multiline image at a frame rate equivalent to a second multiple non-overlapping multiline, wherein the second multiple is a multiple different from the first multiple. In the overlap multiline image, the receive beams of a first ensemble overlap, in a predetermined manner, receive beams of an adjacent ensemble. The second multiple is a multiple less than the first multiple. In one embodiment, the first multiple comprises four times (4×) multiline and the second multiple comprises three times (3×) multiline. Each sequence of an ensemble includes outer round-trip beams.

In addition, the means for implementing the multiline beamforming includes a beamformer configured to arrange corresponding ones of the outer round-trip beams of adjacent ensembles to occur along a same direction; and means for combining sample correlations derived from the outer round-trip beams of the adjacent ensembles along the same direction.

In one embodiment, the beamformer is configured to interleave corresponding ones of the outer round-trip beams of a first ensemble with corresponding ones of the outer round-trip beams of a second ensemble adjacent the first ensemble, for example, by an interleaving factor of two (2). Note that use of the interleave factor of two (2) herein is merely an example. The techniques of the present disclosure can apply to any interleave factor. In addition, the beamformer is configured to not interleave corresponding ones of the outer round-trip beams of a second ensemble with corresponding ones of the outer round-trip beams of a third ensemble adjacent the second ensemble. Still further, the beamformer is configured to interleave corresponding ones of the outer round-trip beams of a third ensemble with corresponding ones of the outer round-trip beams of a fourth ensemble adjacent the second ensemble, for example, by an interleaving factor of two (2).

The system further comprises means for processing ensemble data for outer round-trip beams separately for each ensemble. The processing means processes the ensemble data for outer round-trip beams of a first ensemble having a first transmit direction separately from ensemble data for outer round-trip beams of a second ensemble having a second transmit direction. The system further comprises means for performing clutter filtering and sample correlation with no mixing of data from different transmit directions of respective ensembles. Clutter filtering and sample correlation are performed with no mixing of data from different transmit directions and includes processing outer beams of ensemble data for a first transmit direction separately from outer beams of ensemble data for a second transmit direction, the second transmit direction being different from the first transmit direction.

According to another embodiment, the means for implementing multiline beamforming is further configured to change a (beamforming) transmit direction between successive frames of ensembles, wherein each frame includes of a predetermined number of ensembles. Transmit directions of a current frame are configured to occur where the outer round-trip beams of a previous frame occur.

According to another embodiment, an apparatus comprises: a display; a computer/control unit coupled to the display, wherein the computer/control unit provides data to the display for rendering a screen view; and means coupled to the computer/control unit for providing inputs to the computer/control unit, wherein the computer/control unit is programmed with instructions, responsive to said input means, for carrying out the method of multiline ultrasound imaging as discussed herein.

Still further, according to yet another embodiment, a computer program product comprises computer readable media having a set of instructions that are executable by a computer for carrying out the method of multiline ultrasound imaging as discussed herein.

Although only a few exemplary embodiments have been described in detail above, those skilled in the art will readily appreciate that many modifications are possible in the exemplary embodiments without materially departing from the novel teachings and advantages of the embodiments of the present disclosure. In particular, the embodiments discussed herein can be extended to operate with interleave factors greater than two (2). For example, the embodiments of the present disclosure can be applied to any application involving ultrasound medical imaging systems. Accordingly, all such modifications are intended to be included within the scope of the embodiments of the present disclosure as defined in the following claims. In the claims, means-plus-function clauses are intended to cover the structures described herein as performing the recited function and not only structural equivalents, but also equivalent structures.

In addition, any reference signs placed in parentheses in one or more claims shall not be construed as limiting the claims. The word “comprising” and “comprises,” and the like, does not exclude the presence of elements or steps other than those listed in any claim or the specification as a whole. The singular reference of an element does not exclude the plural references of such elements and vice-versa. One or more of the embodiments may be implemented by means of hardware comprising several distinct elements, and/or by means of a suitably programmed computer. In a device claim enumerating several means, several of these means may be embodied by one and the same item of hardware. The mere fact that certain measures are recited in mutually different dependent claims does not indicate that a combination of these measures cannot be used to an advantage.

1. A method for multiline ultrasound imaging, comprising:
implementing multiline beamforming with a number of ensembles, each ensemble including a sequence of transmit beams of a given transmit direction and a first multiple of receive beams per transmit beam; and
constructing an overlap multiline image at a frame rate equivalent to a second multiple non-overlapping multiline, wherein the second multiple is a multiple different from the first multiple.

2. The method of claim 1, wherein the receive beams of a first ensemble overlap, in a prescribed manner, receive beams of an adjacent ensemble.

3. The method of claim 1, wherein the first multiple comprises four times (4×) multiline and the second multiple comprises three times (3×) multiline.

4. The method of claim 1, wherein each sequence of an ensemble includes outer round-trip beams, further wherein implementing the multiline beamforming includes:
configuring corresponding ones of the outer round-trip beams of adjacent ensembles to occur along a same direction; and
combining sample correlations derived from the outer round-trip beams of the adjacent ensembles along the same direction.

5. The method of claim 4, wherein configuring corresponding ones of the outer round-trip beams of adjacent ensembles to occur in the same direction further includes interleaving corresponding ones of the outer round-trip beams of a first ensemble with corresponding ones of the outer round-trip beams of a second ensemble adjacent the first ensemble.

6. The method of claim 5, wherein the interleaving includes interleaving by a factor of two (2).

7. The method of claim 5, wherein configuring corresponding ones of the outer round-trip beams of adjacent ensembles to occur in the same direction still further includes not interleaving corresponding ones of the outer round-trip beams of a second ensemble with corresponding ones of the outer round-trip beams of a third ensemble adjacent the second ensemble.

8. The method of claim 7, wherein configuring corresponding ones of the outer round-trip beams of adjacent ensembles to occur in the same direction still further includes interleaving corresponding ones of the outer round-trip beams of a third ensemble with corresponding ones of the outer round-trip beams of a fourth ensemble adjacent the second ensemble.

9. The method of claim 4, further comprising:
processing ensemble data for outer round-trip beams separately for each ensemble.

10. The method of claim 9, wherein the ensemble data for outer round-trip beams of a first ensemble having a first transmit direction is processed separately from ensemble data for outer round-trip beams of a second ensemble having a second transmit direction.

11. The method of claim 9, further comprising:
performing clutter filtering and sample correlation with no mixing of data from different transmit directions of respective ensembles.

12. The method of claim 11, wherein the performing clutter filtering and sample correlation with no mixing of data from different transmit directions includes processing outer beams of ensemble data for a first transmit direction separately from outer beams of ensemble data for a second transmit direction, the second transmit direction being different from the first transmit direction.

13. The method of claim 1, further comprising:
adjusting beamforming parameters to ensure that a frame rate of the multiline beamforming is adequate to observe hemodynamics in a region of interest.

14. The method of claim 1, further comprising:
changing a beamforming transmit direction between successive frames of ensembles, wherein each frame includes of a predetermined number of ensembles.

15. The method of claim 14, wherein transmit directions of a current frame are configured to occur where the outer round-trip beams of a previous frame occur.

16. A system for multiline ultrasound imaging, comprising:
means for implementing multiline beamforming with a number of ensembles, each ensemble including a sequence of transmit beams of a given transmit direction and a first multiple of receive beams per transmit beam; and
means for constructing an overlap multiline image at a frame rate equivalent to a second multiple non-overlapping multiline, wherein the second multiple is a multiple different from the first multiple.

17. The system of claim 16, wherein the receive beams of a first ensemble overlap, in a predetermined manner, receive beams of an adjacent ensemble.

18. The system of claim 16, wherein the first multiple comprises four times (4×) multiline and the second multiple comprises three times (3×) multiline.

19. The system of claim 16, wherein each sequence of an ensemble includes outer round-trip beams, further wherein said means for implementing the multiline beamforming includes:
a beamformer configured to arrange corresponding ones of the outer round-trip beams of adjacent ensembles to occur along a same direction; and
means for combining sample correlations derived from the outer round-trip beams of the adjacent ensembles along the same direction.

20. The system of claim 19, further wherein the beamformer is configured to interleave corresponding ones of the outer round-trip beams of a first ensemble with corresponding ones of the outer round-trip beams of a second ensemble adjacent the first ensemble.

21. The system of claim 20, wherein the beamformer is configured to interleave by an interleaving factor of two (2).

22. The system of claim 20, further wherein the beamformer is configured to not interleave corresponding ones of the outer round-trip beams of a second ensemble with corresponding ones of the outer round-trip beams of a third ensemble adjacent the second ensemble.

23. The system of claim 22, still further wherein the beamformer is configured to interleave corresponding ones of the outer round-trip beams of a third ensemble with corresponding ones of the outer round-trip beams of a fourth ensemble adjacent the second ensemble.

24. The system of claim 19, further comprising:
means for processing ensemble data for outer round-trip beams separately for each ensemble.

25. The system of claim 24, wherein said processing means processes the ensemble data for outer round-trip beams of a first ensemble having a first transmit direction separately from ensemble data for outer round-trip beams of a second ensemble having a second transmit direction.

26. The system of claim 24, further comprising:
means for performing clutter filtering and sample correlation with no mixing of data from different transmit directions of respective ensembles.

27. The system of claim 26, wherein performing clutter filtering and sample correlation with no mixing of data from different transmit directions includes processing outer beams of ensemble data for a first transmit direction separately from outer beams of ensemble data for a second transmit direction, the second transmit direction being different from the first transmit direction.

28. The system of claim 16, wherein said means for implementing multiline beamforming is further configured to change a beamforming transmit direction between successive frames of ensembles, wherein each frame includes of a predetermined number of ensembles.

29. The system of claim 28, wherein transmit directions of a current frame are configured to occur where the outer round-trip beams of a previous frame occur.

30. An apparatus comprising:
a display;
a computer/control unit coupled to the display, wherein the computer/control unit provides data to the display for rendering a screen view; and
means coupled to the computer/control unit for providing inputs to the computer/control unit, wherein the computer/control unit is programmed with instructions, responsive to said input means, for carrying out the method of multiline ultrasound imaging as claimed in claim 1.

31. A memory comprising a computer program product comprising computer readable media having a set of instructions that are executable by a computer for carrying out the steps of:
a computer-readable medium for implementing multiline beamforming with a number of ensembles, each ensemble including a sequence of transmit beams of a given transmit direction and a first multiple of receive beams per transmit beam; and
a computer-readable medium for constructing an overlap multiline image at a frame rate equivalent to a second multiple non-overlapping multiline, wherein the second multiple is a multiple different from the first multiple.
